(19) United States
(12) Patent Application Publication
(10) Pub. No.: US 2024/0355482 A1
(43) Pub. Date: Oct. 24, 2024

(54) Measuring Mitochondrial Balance and Resilience

(71) Applicants: Debra Strick Rivera, Los Angeles, CA (US); Dahabada Helena Jose Lopes, Topanga, CA (US); Elisa Clay, Topanga, CA (US); Deborah Klowden Mann, Culver City, CA (US)

(72) Inventors: Debra Strick Rivera, Los Angeles, CA (US); Dahabada Helena Jose Lopes, Topanga, CA (US); Elisa Clay, Topanga, CA (US); Deborah Klowden Mann, Culver City, CA (US)

(21) Appl. No.: 18/676,394

(22) Filed: May 28, 2024

(51) Int. Cl. G16H 50/30; G16H 10/20; G16H 10/40; G16H 10/60; G16H 20/10; G16H 50/70

(52) U.S. Cl. CPC G16H 50/30; G16H 10/20; G16H 10/40; G16H 10/60; G16H 20/10; G16H 50/70

(57) ABSTRACT

The invention is a method for assessing health status and changes thereof due to interventions. Mitochondrial imbalance is a common feature across diseases. Mitochondrial balance can be inferred from cellular metabolism including the processing of physiological stress messengers, both of which are modulated by prolonged stress. As such, measures of glutathione redox status, metabolic status, and stress measures, can be used to infer the health status, resilience to stress, and risk of an individual or population, as well as to inform efficacy of an intervention for improving health status. The approach applies to mental health, neurological disorders, disease, inflammation, organ dysfunction, personalized treatment assessment, as well as policy making and design strategies that promote wellbeing.

### SPECIFICATION CROSS REFERENCE TO RELATED APPLICATION

Cross-reference to application Ser. No. 18/675,621

### STATEMENT REGARDING FEDERALLY SPONSORED RESEARCH OR DEVELOPMENT

Not Applicable

### FIELD

The present invention proposes the measurement and tracking of cellular metabolism, as an indicator of health and disease status. We define health as resilience to stress, and identify the modulation of metabolism and the systems that maintain redox balance, and process reactive oxygen species, as key factors for health. Mitochondria are central to a dynamic system that balances the needs of the cell, organ, organism, and individual. Direct and indirect measures of mitochondrial function can be used as key indicators of metabolic balance, changes thereof, and inform decisions to promote health and wellbeing. Metabolic imbalance is a shared common feature across diseases, and presents as a mechanism by which stress modulates health, disease, and ultimately the rate of aging. A key embodiment, the quantification of the ratio of the reduced glutathione (GSH) to oxidized glutathione (GSSG) in the blood serum of humans is a direct measurement of a redox couple that counteracts the buildup of ROS, and as such can be used as to inform and monitor health status. As this is a biosystems approach use cases span tissue, organs, functional systems (e.g., cardiovascular, hormonal, immunological), individuals and larger communities or populations. The herein described methods apply to a diverse range of topics, including but not limited to: informing health status or risk of an individual, evaluating efficacy of a treatment, measurement-based dosing, well-being enhancing design, quantifying health and health disparity of a population, and guiding public policy to target public well-being.

### BACKGROUND

Mitochondria are the hub of the eukaryotic cell and by extension, the organism. Traditionally thought of as the “power house of the cell,” there is an abundance of evidence in the scientific literature that the mitochondria orchestrate the critical processes of a cell through a dynamic interplay of flux through metabolic pathways. Metabolism encompasses the biochemical processes of an organism. This includes but is not limited to: protein synthesis and breakdown, energy production, glucose metabolism, hormone synthesis and break down, neurotransmitter synthesis and break down, regulation of gene expression, carbohydrate synthesis and breakdown, nutrient signaling, lipid and membrane synthesis and breakdown, DNA synthesis, damage, and repair, as well as cell proliferation, differentiation, repair, necrosis, and death. Many things modulate metabolism and are in turn modulated by metabolic processes, including the physiological stress response, toxins, hormones, inflammation, immune response, mood and also neurotransmitters. The entire set of processes are interdependent, forming a dynamic system. Mitochondrial health is gaining momentum as a common bio-mechanism across aging and diseases, including neurodegeneration, mental illness, cardiovascular disease, infection, brain injury, cancer proliferation and drug resistance, organ failure, and metabolic maladies. Mitochondrial health, while crucial to the overall health of the organism, has hitherto been difficult to measure, being either invasive, costly, or both. There are many ways to enter a systemic state of deteriorated mitochondrial health, and equally many ways to support an individual in exiting such a state.

Maintaining mitochondrial health requires not just the efficient processing of metabolic substrates, but also defense against redox imbalance and oxidative stress. This intricate balance ensures that mitochondria can effectively meet the cellular energy demands while maintaining structural and functional integrity. Through the Krebs cycle, critical electron carriers, NADH and FADH2, capture energy in the form of high-energy bonds, driving the proton gradient that powers ATP synthesis. The Kreb's Cycle, takes place in the mitochondria and generates not only ATP, but also produces an abundance of reactive oxidative species (ROS). ROS is a key signaling mechanism for metabolism and stress response, with roles across scales from molecular to behavioral. For example, when an organism is fighting a viral or bacterial infection, the ROS both initiates an immune response, including the triggering of inflammation, and also will cause the organism to alter behaviors to conserve energy. The systems and pathways involved in ROS regulation include the glutathione system, methionine pathway, the folate cycle, the pentose phosphate pathway, and the thioredoxin system. When the ROS accumulate, the Kreb's cycle is blocked evidenced by an increase in succinate concentration in the mitochondria. The increased succinate signals the release of hypoxia inducing factor one alpha (HIF1alpha), triggering inflammation and glycolysis. The higher the baseline ROS concentration is in the mitochondria, the lower the threshold is for triggering inflammation. When ROS signaling does not dissipate in mitochondria damage ensues. Accumulating ROS causes mitochondrial DNA (mtDNA) mutation, Electron Transport Chain (ETC) impairment, lipid peroxidation of mitochondrial membranes, oxidative modifications of mitochondrial proteins, the release of cytochrome c triggering apoptosis, dysregulation of calcium homeostasis, further activation of inflammatory responses, and increased autophagy and mitophagy to remove damaged mitochondria.

Maintaining the redox balance of the cell is critical for mitochondrial health. Cells maintain their redox balance through several key redox-coupled pairs, which are sets of molecules that can undergo reversible oxidation and reduction reactions. These pairs are crucial in buffering the cell against redox imbalance, including oxidative stress, and in regulating various cellular processes. In terms of mitochondrial health, important redox-coupled pairs include the Glutathione (GSH/GSSG) Pair, Nicotinamide Adenine Dinucleotide Pairs (NAD⁺/NADH) and NADP⁺/NADPH), the Thioredoxin (Trx(SH)2/TrxSS) System, Peroxiredoxin/Sulfuredoxin System, Ascorbate/Dehydroascorbate Pair, and the Ferric/Ferrous Iron Couple.

In healthy tissue glutathione plays a fundamental part in maintaining the redox balance of the cell, preventing the ROS from accumulating. Glutathione is a tripeptide in reduced form, comprising of glycine, glutamate, and cysteine amino acid residues. The glutathione system has a fundamental role in all eukaryotic cells of maintaining the homeostasis of mitochondria. This role extends beyond energy production, as all biological stressors lead to increased reactive oxidative species. This includes but is not limited to exogenous toxin exposure, stress, blunt trauma, infection, sleep disturbance, social stresses, cardiovascular events, and glucose spikes.

Depending on the redox status of tissue, proteins, for example p53, have differential actions. In the case of p53, when a stressor falls on reduced tissue, p53 supports the glutathione system. However, when a stressor falls on oxidized tissue, e.g., hypoxic conditions, p53 acts to promote hypoxic and inflammatory conditions by stabilizing the hypoxia inducing factor one alpha (HIF1 alpha; 10.1093/nar/gkz766). Telomere length is also modulated by cellular redox status and p53. Telomere length is a known biomarker of age and longevity, and is reduced by redox imbalance (e.g. oxidative stress) and inflammation (10.2174/1874609808666150122153151). Biomarkers of aging have predictive value, as in U.S. Pat. No. 11,193,169 B2 using the metric of average telomere length to predict risk of mortality. Telomere length has been shown to have statistically significant correlation with biological age normalized GSH:GSSH of the blood (10.1089/rej.2021.0045). Similarly, accumulation of altered methylation is being used as a biological clock. Methylation is modulated by low reduced glutathione pools and/or ROS and reactive nitrogen species build up due to allosteric load. It is possible that increased changes in methylation, termed epigenetic drift, is an indirect measure or history of the degree and extent to which a given cell has been unable to maintain redox balance, for which reduced glutathione stores play a critical role.

The glutathione system, creates positive feedback loops that are either anti-inflammatory when reduced glutathione is dominant, and inflammatory when reduced glutathione is deficient. A healthy level of reduced glutathione helps to maintain optimal functioning of Nuclear factor erythroid 2 (Nrf2). Nrf2 in turn supports expression of enzymes involved in glutathione synthesis and recycling, which enhances cellular antioxidant capacity. Nrf2 also inhibits Nuclear Factor kappa-light-chain-enhancer of activated B cells (NF-kappa B). Under stressful conditions (allosteric load), the NF-kappa B pathway releases pro-inflammatory cytokines including tumor necrosis factor alpha (alpha). TNF alpha when elevated for extended periods, as it is with chronic stress, depletes reduced glutathione stores and also suppresses Nrf2 expression, further worsening the low levels of glutathione and making the cells less resilient to oxidative stress. As such, a state of glycolysis and a shift away from Krebs cycle respiration can persist. In such a manner the allostatic pressure of persistent stress, shifts the metabolic and redox balance of the cell, organ, or organism to a new state. This persistent stress adaptive state, termed allostatic load or overload, can cause metabolic, cognitive, hormonal, mood/mental health, cardiovascular, inflammatory, and immune consequences.

Replenishing glutathione can help redox balance, allowing the return towards a more dynamic metabolic state in which Krebs cycle, rather than glycolysis becomes the prominent respiration pathway, in turn reversing consequences associated with allostatic (over) load. There are diverse ways in which endogenous glutathione stores can be replenished and supported. For example, U.S. Pat. No. 11,730,711 B2 from mid 2023 presents the use of NAC for increasing endogenous glutathione levels. Supplementation with glutathione has been shown to restore microbiome balance (10.1093/femsle/fnad116, 10.1038/s41598-020-64834-3). Life-style interventions such as diet, exercise, improved sleep, social engagement, time in nature, emotional support, experiencing awe, and other stress management techniques can replenish depleted glutathione stores. Examples in the medical literature show increases in reduced glutathione and cysteine in the serum, decreased inflammatory cytokines, and improved reported mood and resilience from activities such as yoga and mindfulness meditation (10.1016/j.bbih.2021.100372, 10.1089/acm.2007.0567). The complex interplay of tissue redox balance, hormones, inflammation, glucose metabolism, cardiovascular health, pain, the hypothalamus pituitary adrenal axis and immune system dysregulation can be difficult to target. However, supporting the glutathione system, which in turn supports Nrf2 in upregulating various antioxidant systems, can aid the return to a healthier baseline.

### BRIEF DESCRIPTION OF THE INVENTION

Herein we propose the use of metabolic status as a means of indicating health status and overall wellbeing. All established longevity measures, e.g., telomere length and epigenetics (e.g. gene expression and methylation) are indirect measures of redox status, mitochondrial health, and even the glutathione system. From the perspective of process as the fundamental unit of life, metabolism emerges as central to health and well-being. Mitochondria serve as a central hub of metabolism, responding to the needs of the organism to balance the energetic and material needs. Gene expression, protein folding, hormone expression, neurotransmitter flux, and immune response are all tightly controlled by the mitochondria. Mitochondria are very sensitive to stress, and allow the body to adapt to changing conditions both internally and in the environment. The mitochondrial-centric perspective yields a biosystems approach, in which metabolic processes and tissue redox status are intertwined. Whether as an upstream or downstream mechanism, or both, as is more consistent with a systems biology perspective, oxidative stress and mitochondrial health, are implicated across diseases. This is consistent with observations of cross-correlation across disease states, as well as the influence of stress and environment on overall health. Whereas epigenetics, and proteomics are widely variable across individuals and diseases, mitochondrial health is a shared biomechanism across diseases and is a commonality across all eukaryotes. Redox balance and ROS flux are key modulators of metabolism and determinants of mitochondrial health. Thus, the systems and pathways that modulate ROS production and clean-up also present as key factors for health and wellbeing. Again, we have a feedback system. Add further that ROS build up can impede processes by which dysfunctional cells or organelles are metabolized, and can lead to further build-up of ROS. Likewise, accumulation of ROS can lead to conditions in which chronic inflammation and further ROS homeostasis disruption. As ROS is a key signal across stressors, mitochondria and the key ROS homeostasis regulating support pathways can be seen as systems that give resilience to stress. Health in turn, can be seen as the resilience to stress. As a surrogate to classical endpoints such as survival or disease incidence, measures of mitochondrial health, including the pathways and systems that metabolize ROS, have the potential to enhance tailored treatments and treatment development for chronic and acute disease. With implications for mental health and resilience to stress, mitochondrial health is crucial for overall wellbeing. As such, policy and design choices that support wellbeing can be informed and streamlined by measuring the direct or indirect effect on mitochondrial health.

The herein invention, recognizing the position of mitochondria as a metabolic hub and metabolism as central to health, opens up a multitude of options for monitoring mitochondrial health via the modulators, products, reactants, and intermediaries of mitochondrial function. Stress hormones, sex hormones, metabolic peptides (e.g., insulin, amylin, glucagon, ghrelin), enzymes, signaling molecules, metabolites, products of lipid and protein catabolism, and physiological signals can be used as indirect measures of mitochondrial function. Individual derived metrics capturing energy level, pain and inflammation level, allergic response, sleep quality, breast tenderness, mood and dysregulation can all be interpreted in the context of mitochondrial health. While mitochondrial health can be measured directly from biopsied (ex vivo) materials, invasive procedures present with added cost, risk, and complexity. Though net yet used clinically, blood serum measures of glutathione redox status are a sensitive measure of mitochondrial health across all disease categories and a diverse range of health and lifestyle interventions. Making the connection that mitochondrial health is central to health and wellbeing, blood serum measures of glutathione redox status become a quantitative measure of health status, disease risk, well-being and changes thereof. In turn, modulators of mitochondrial health, function, and the glutathione system present additional measures for health and well-being. Likewise, metabolic pathways yield a multitude of measures of mitochondrial health, and therefore appropriate indicators of health and wellbeing. Stress, is a key modulator of metabolism and, when prolonged degrades mitochondrial health. Ergo physiological measures of stress provide indirect metrics for mitochondrial health. A series of symptoms and metabolic processes are associated with chronic stress and ROS accumulation, and provide measures of mitochondrial health. The glutathione system, telomere length, and methylation patterns hold information about cumulative mitochondrial strain over a lifetime. Optimal immune functioning and gut microbiome are also indirect measures of mitochondrial health and therefore indicators of health status and wellbeing. Physiological measures of stress can inform whether presented stimuli support or impair mitochondrial health, and therefore health and well-being. Sensitivity, stability, time-frame, cost, and convenience can be factors for choosing one or more measures of mitochondrial health for a given use case. Spectral patterns in heart-rate variability and mutations in mitochondrial DNA both inform mitochondrial health, and are therefore tools for assessing health status and wellbeing. The applications of this approach are wide in scope.

The search for biomarkers of disease and longevity rely on the underlying assumption that there will be uniformity across individuals that fall into the same category. Typically, clinical data is analyzed with machine learning for emerging patterns and statistical significance. However, the interplay between the biological processes of an individual is inherently non-linear and leads to wide-variation across a population. Even if the population is comprised of individuals sharing a single diagnosis, the adaptive nature of metabolism and the heterogeneity of genes and gene expression are further compounded by confounding variables such as sleep, history of infection or other trauma, emotional state, stress, pain level, nutrition, and exercise. Such an approach also requires the exclusion of individuals with more than one disease. However, the data has revealed that having one disease or disorder makes a person more likely to accumulate more. It is not uncommon for a person with an autoimmune disorder or diabetes to collect additional diagnoses over time.

The present invention is a biosystems approach. The key insight is that the mitochondria is a hub of the dynamic system, acting as a sensor, actuator, and central modulator of metabolism and overall health. Rather than attempting to capture repeatable patterns from an inherently dynamic and heterogeneous system, we create a tool kit of indicators of mitochondrial health. It is still within reason to employ machine learning to select the optimal set of measures of mitochondrial health for a given use case, however the approach will inherently be influenced by the dataset presented and the selection of markers specifically linked to mitochondrial health. Therefore, taking as a starting point the premise that mitochondrial health informs overall health, the outcome is inherently different from standard approaches to sifting through clinical data to find useful biomarkers.

A biomarker presents not only an informative measurement, but also a target for health interventions. Interestingly, conventional wisdom for supporting health and well-being leads to a great starting point for supporting mitochondrial health: prioritizing sleep, exercise, stress-management, time outdoors and in nature, social connection, and a nutrient rich diet. Going a step further, it is possible to support metabolic pathways that support mitochondrial health, including but not limited to the glutathione system and the pentose phosphate pathway.

Efforts to rectify health disparities have been limited by a lack of a clear or unifying biomechanism. Mitochondrial health, and for example the glutathione system are measurable and actionable targets for health disparity. Mental health, cardiovascular disease, metabolic health, immune health, allergic response, hormone balance, joint health, inflammation, nerve pain, cognitive state, and neurodegenerative disease are all intertwined with mitochondrial health. Stress and environmental toxins present as burdens on the glutathione system, leaving mitochondria vulnerable to oxidative damage. Insulin resistance, inflammation, hormone dysregulation, impaired immune function, DNA damage, accelerated aging, and reduced ability to repair tissue may emerge when oxidative damage persists. Such a situation creates a feedback cycle in which impaired health, leads to increased allosteric pressure, further metabolic dysfunction, and diseases accumulation. Across the board, the diseases associated with health disparity, from glaucoma to maternal mortality are linked to low reduced glutathione levels.

To further illustrate the scope of the invention we present a series of embodiments. As a first exemplary embodiment, the herein described approach is used in a series of embodiments in order to determine the efficacy of a given intervention in a single individual or in a group of individuals. As baseline values of glutathione, or the ratio of reduced to oxidized glutathione can very across individuals, such an approach of characterizing the change from baseline in a given individual can overcome the confounding variable of intra-individual variation in baseline. In one embodiment, one or more individuals is given a dietary supplement derived from cruciferous vegetables, sulforaphane, known to both promote reduced glutathione stores and also Nrf2. Prior to being given the supplement, the participant(s) undergo glutathione-uptake mapping as has been described in affiliated patent application Ser. No. 18/675,621.

After a period of time, e.g., 6 weeks, the participant(s) return to repeat the glutathione-uptake mapping procedure to identify regions in which the sulforaphane dosage was sufficient to overcome glutathione deficiency. If there is no change in glutathione uptake, it can be inferred under the assumption that no other variables were altered (e.g., no change in stress level, sleep quality, exercise, diet, infection, medication, etc.) that the sulforaphane was not effective for this dose and duration for this individual. On the other hand, if the uptake maps indicate that one or more areas uptake less glutathione and the aforementioned assumption holds, it can be inferred that the glutathione deficiency in those regions has been reduced by the sulforaphane regiment. If some regions do not show reduced uptake, an increase in dose and/or duration of the sulforaphane regiment, or supplementation with additional interventions, including but not limited to dietary or life-style changes, may result in further improvements. Further embodiments replacing sulforaphane with life-style, pharmaceutical, dietary/nutraceutical, or even public policy, environment, or services can be imagined.

In another embodiment, oral liposomal “Trizomal” glutathione (Apex Energetics, Irvine, California) is administered to individuals for the purpose of informing glutathione deficiency or the need for glutathione system support, based on self-reported symptom changes. This glutathione formulation, designed for enhanced absorption and bioavailability, encapsulates glutathione within a liposomal delivery system, facilitating its direct uptake by the cells. Upon administration, individuals will monitor and report changes in symptoms associated with glutathione deficiency, such as chronic fatigue, weakness, or cognitive issues. The efficacy of the liposomal “Trizomal” glutathione is assessed through these self-reported experiences, tracking improvements in symptoms over a specified period. This approach provides a practical, non-invasive method to determine the necessity and effectiveness of glutathione supplementation in individuals potentially suffering from a deficiency or those who may benefit from enhanced glutathione system support. The self-reported outcomes will guide further recommendations for ongoing glutathione therapy, offering a tailored approach to addressing oxidative stress and its associated conditions.

In a further embodiment, consider a company that provides as a service to individuals data to help them evaluate if the life-style or treatment protocols that they are using are helping them to meet their health goals. By monitoring GSH:GSSG and or related data metrics prior to and after a period of initiating the treatment, the individual can gain data to inform the cost-benefit analysis of the intervention. While GSH:GSSG is used in research, to our knowledge it is not yet being offered to consumers.

In a clinical setting, the use of GSH:GSSG or similar metrics are also not in use. However, by evaluating resilience to stressors, this provides a per individual means of adjusting risk factors. In one such embodiment, by using GSH:GSSG, glutathione systems enzymes, or a marker such as Nrf2, the clinicians can then assess the overall vulnerability of the patient instead of using factors such as age and gender. This informs the vulnerability or protection against ferroptosis, which is a common pathway for cardiac and brain cell apoptosis. Here we depart from population statistics in favor of personalized risk assessment in the clinic, presurgical evaluation, and in emergency medical care. This embodiment is also of use to determine risk for cognitive impairment or neurological vulnerability after neurological traumas including stroke and physical trauma. Similarly, the approach is useful for informing risk of pregnancy complications, such as preeclampsia, which has been linked to glutathione deficiency in the placenta.

Another embodiment of the invention involves monitoring and addressing glutathione deficiencies linked to conditions such as post-concussion syndrome, post-viral sequelae, PTSD, dysautonomia, and stroke. Appropriate monitoring measures include, but are not limited to measuring serum ratio of reduced glutathione (GSH) to oxidized glutathione (GSSG) in the body, and/or evaluating self-reported symptoms such as energy levels or irritability. These assessments establish a baseline and enable follow-up tracking to gauge the progression or improvement of the glutathione deficiency. Interventions for individuals identified with glutathione deficiencies are varied and can be personalized based on individual needs and the specific condition being treated. These interventions may include, but are not limited to, the administration of N-acetylcysteine (NAC), NAC ethyl-ester (NACET)), oral glutathione supplements, targeted micronutrient supplementation, such as Vitamin D, selenium, Vitamin B complex, and Zinc, each known to play a role in maintaining or enhancing glutathione levels and overall antioxidant status. Additionally, lifestyle interventions are considered essential components of the treatment plan. These may encompass regular exercise, optimized sleep patterns, meditation, social engagement, and emotional processing strategies. Nutritional interventions that support mitochondrial health can play a key role, including but not limited to eating regularly, prioritizing protein over carbohydrates and processed food, and limiting daily sugar intake to less than 12 grams per day. This holistic approach not only aims to rectify glutathione imbalances but also addresses the broader spectrum of physiological and psychological symptoms associated with the aforementioned conditions. By regularly monitoring GSH:GSSG ratios and self-reported symptoms, the efficacy of these interventions can be evaluated, and treatment plans can be adjusted as needed.

Similarly, another embodiment can be used to mitigate the health consequences of toxin exposure, including but not limited to Per- and Polyfluoroalkyl Substances (PFAS). In this case, a baseline blood measure for PFAS can be included prior to beginning support of the glutathione system. Glutathione is capable of conjugating with toxins, including PFAS, facilitated by the enzyme glutathione s-transferase, and then is subsequently eliminated. Decreases in GSH are reported in cell culture studies of PFAS exposure. Supplementing glutathione may help mitigate the health consequences of PFAS through helping to restore redox balance. Environmental toxin exposure is also known to present as a health disparity. The increased environmental toxin exposure can deplete reduced glutathione pools and lead to a myriad of compounding health consequences. In this embodiment, an appropriate quantitative measure can include, but is not limited to, cytosol or serum levels of PFAS, and serum, or red blood cell measures of GSH:GSSG. The treatment in this embodiment can be anything that supports the glutathione system or Nrf2. Candidate interventions include, though are not limited to, exogenous or glutathione precursors.

A further embodiment of the presented invention is for the targeting of health disparities of marginalized communities. Supporting the glutathione system as a means of overcoming health disparities has innumerable use cases, including but not limited to at risk women preparing for child bearing, individuals with multiple chronic diseases, people that are prediabetic or diabetic, and those exposed to toxin exposure, including but not limited to nuclear radiation and toxic spills. Programs that offer exogenous glutathione or glutathione precursors can be offered to individuals that present with low baseline GSH:GSSG. Similarly, monitoring of GSH:GSSG over time for a given community, in for example a toxic spill zone, can be of use to determine candidates for intervention as well as comparison of the levels in those choosing to participate in the intervention as compared to those that are not participating. Over time, a body of data may be generated to guide public policy for remediation of at risk communities.

In a further embodiment measures of mitochondrial health can be used for tailoring doses to an individual. This takes a biosystems approach to fine-tune standard dosing that is conventionally determined via population statistics. Hormonal balance, an indicator of mitochondrial health, and/or glutathione levels can be monitored to help ensure that the desired benefits of a treatment are balanced with mitochondrial health to evaluate and minimize long-term consequences of a treatment. This use case has applications that include but are not limited to: hormone replacement therapy, modulation of glucose metabolism, and cardiovascular interventions.

In yet another embodiment, changes in GSH:GSSG blood measures can be used as an endpoint for evaluating efficacy of therapeutic interventions, including but not limited to pharmaceutical and nutraceutical compounds, as well as life-style interventions.

While the potential embodiments of the invention have not fully been captured here, we present a final set of examples in the context of well-being informed design. Environment can improve or impede wellbeing. Light exposure, spaciousness, comfort, safety, awe, elements of nature, colors, sounds, air-quality and flow, and promoting or impeding social engagement can all impact the wellbeing of a person. By measuring directly or indirectly the mitochondrial health, quantitative measures can be derived to inform designs that promote wellbeing. For example, in one approach, an architecture firm can take baseline measures of GSH:GSSG before occupants move into a living or working environment that was designed in a manner that was expected to promote health and wellbeing, and follow up with GSH:GSSG measures to verify the claims and inform design of future spaces. Alternatively, an age and socio-economic matched population could be compared to the population that lives or works in the completed space. In a further embodiment, design elements or simulations can be presented as a stimulus to a cohort of volunteers and measures such as heart rate variability can be characterized before and after exposure to the stimuli. In another such embodiment, a series of candidate design or design elements can be presented and stress-response compared to determine which design is more likely to promote mitochondrial health and wellbeing. The approach can extend to product design, print design, interior design, entertainment, experiential design, and architecture.

### BRIEF DESCRIPTION OF THE DRAWINGS

FIG. 1 illustrates a systematic approach for assessing whether an individual is deficient in glutathione (GSH), beginning first with administering oral liposomal reduced glutathione and seeing if symptoms are reduced.

FIG. 2 depicts the harmful effects of reactive oxygen species (ROS) accumulation within the body, highlighting diseases and aging processes associated with oxidative stress.

FIG. 3 details various factors that can lead to the depletion of glutathione levels in the body.

FIG. 4 enumerates the wide range of functions of mitochondria and the glutathione system, many of which overlap.

FIG. 5 presents various strategies to support mitochondrial health and enhance the glutathione system.

FIG. 6 lists specific markers to differentiate between healthy and impaired mitochondrial function.

FIG. 7 provides a list of diseases associated with mitochondrial dysfunction, categorized by systems affected such as neurological disorders, metabolic issues, and immune system diseases. It highlights the broad impact of mitochondrial health on overall well-being.

FIG. 8 is a non-exhaustive list of tests to assess mitochondrial function and glutathione levels. These include tests for oxidative stress markers, metabolic profiles, and specific tests like glutathione reduced/oxidized ratio (GSH:GSSG) and complete blood counts.

FIG. 9 outlines a method for evaluating the efficacy of a treatment or intervention for an individual. The process begins by defining the treatment or intervention, collecting baseline measurements, administering the treatment over a specified period, and then repeating measurements. The evaluation of changes in these measurements helps to infer the benefit or lack thereof from the treatment.

FIG. 10, similar to FIG. 9, extends to a population scale. This figure details the process of defining a research question, selecting criteria for participant recruitment, obtaining baseline measures, and administering a treatment or intervention. After a period, measurements are repeated, and population statistics are computed to infer evidence for the benefit or no benefit of the treatment across a population.

FIG. 11 depicts a method for wellbeing informed design, specifically using heart rate variability (HRV) as a stress response measure to evaluate different design simulations. The process involves obtaining baseline HRV measures, presenting two design simulation options while monitoring HRV, repeating exposures randomly, and evaluating the data to determine which design option effectively reduces stress. The process is repeated across a population to inform which simulated design reduced the stress measure, guiding the selection of the design that best supports wellbeing.

### DETAILED DESCRIPTION OF THE DRAWINGS

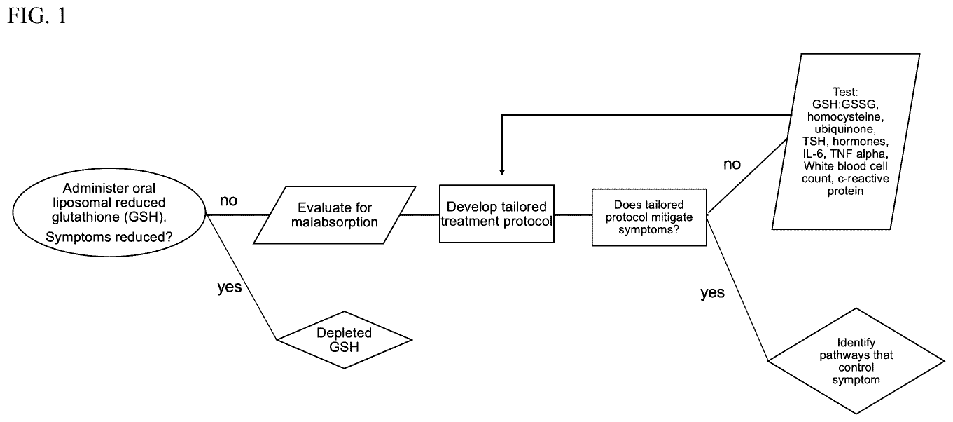

FIG. 1 is a sequential workflow for assessing glutathione (GSH) deficiency in individuals. The process starts with an assessment of symptoms indicative of low GSH levels, such as fatigue, immune dysfunction, or cognitive issues. If the symptoms are reduced by the administration of oral bioavailable GSH, then it can be concluded that the individual has a GSH deficiency. If the symptoms are not alleviated, the next step is to evaluate for malabsorption and micronutrient deficiencies using genetic analysis of relevant single nucleotide polymorphisms (SNP's). Based on the findings, a more tailored treatment is administered. If that treatment fails to alleviate symptoms, further tests are conducted which are not limited to the following though may include one or more: GSH:GSSG, homocysteine, ubiquinone, TSH, Hormones, IL-6, TNF alpha, White blood cell count, glutathione system enzymes, and c-reactive protein. A treatment protocol based on the findings is then given to the individual and iterated until symptoms are alleviated. Through such a process, glutathione system deficiencies can be determined.

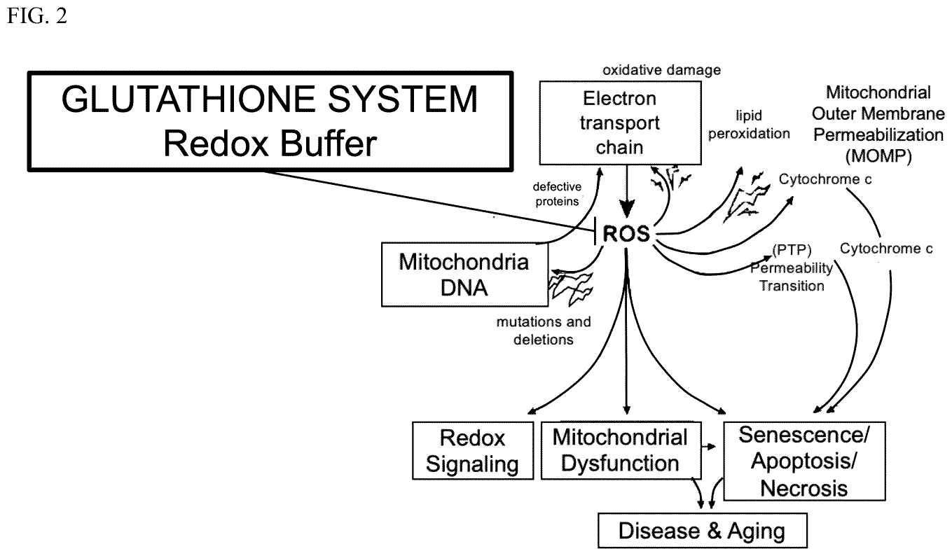

FIG. 2 presents negative impacts of reactive oxygen species (ROS) accumulation on cellular health, illustrating how increased ROS levels contribute to cellular aging and the pathogenesis of various diseases. It details glutathione's role in neutralizing ROS, showcasing pathways such as the detoxification of harmful substances and the repair of oxidative damage to proteins, lipids, and DNA.

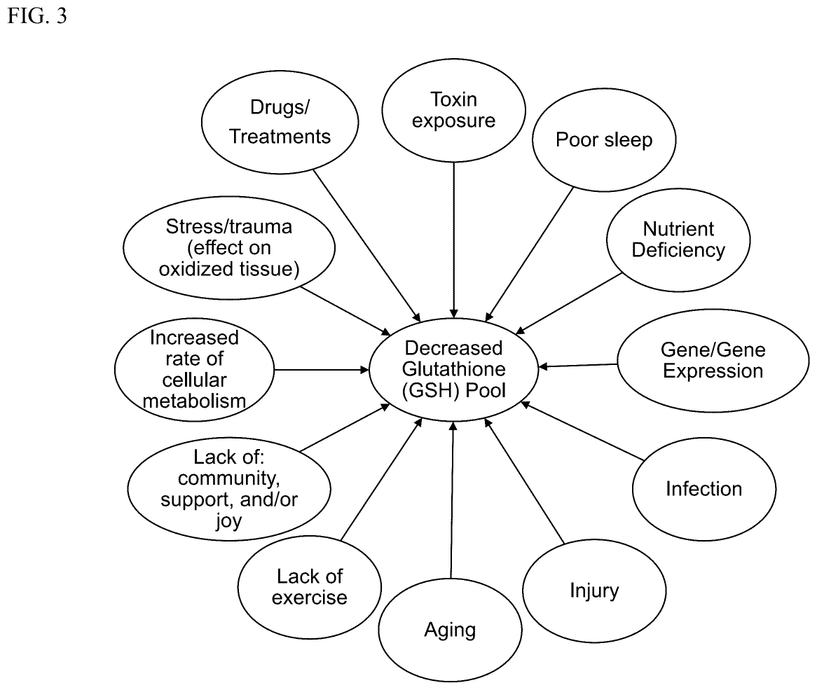

FIG. 3 enumerates various factors that lead to glutathione depletion, including poor diet, pollution exposure, chronic stress, and infections.

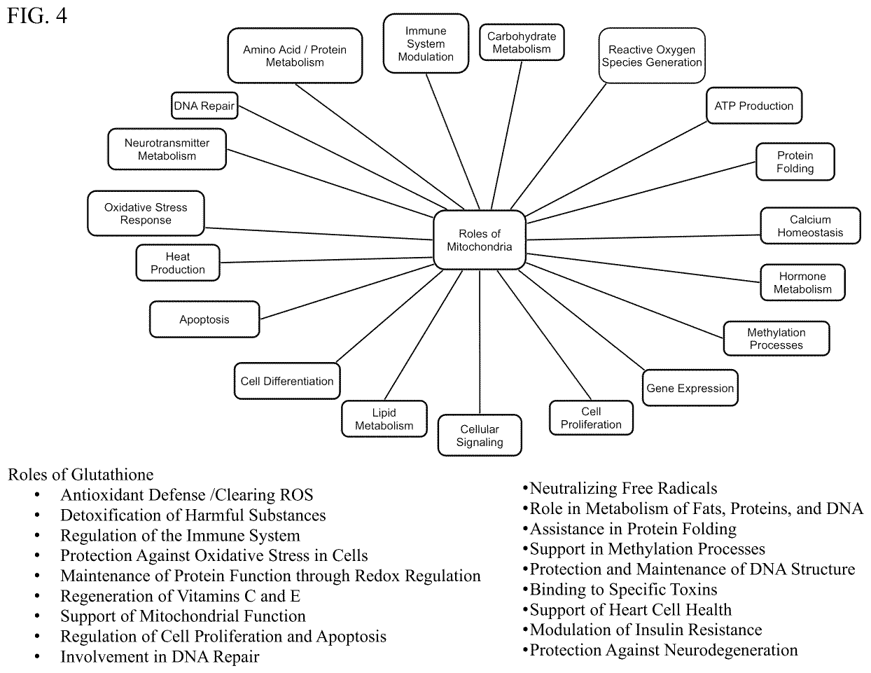

FIG. 4 outlines the broad scope of the roles of mitochondria and glutathione, many of which are overlapping. Mitochondria are involved in synthesis and breakdown of hydrocarbons, lipids, amino acids, proteins, neurotransmitters, and hormones. Mitochondria play a central role in DNA repair and expression, as well as protein folding and methylation. Mitochondria modulate immune system, inflammation, and signaling to activate the hypothalamus pituitary adrenal axis. Cell proliferation, differentiation, and apoptosis are also mediated by the mitochondria and the glutathione system. The glutathione system influence of redox balance and ROS accumulation modulates protein folding, methylation, telomere length, DNA protection and repair, inflammation response, immune system modulation, stress response, balance of metabolic pathways, lipid oxidation, insulin sensitivity, and more.

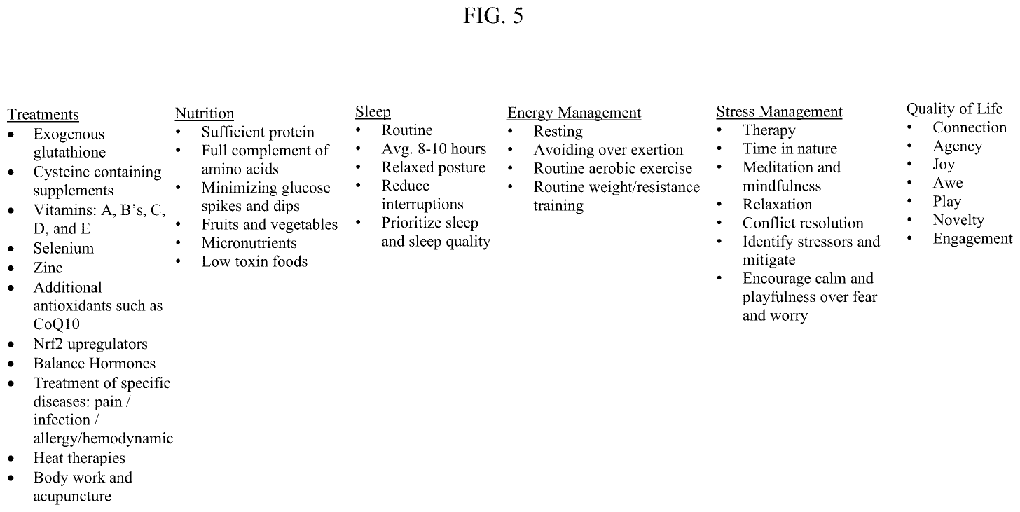

FIG. 5 details the diverse aspects of supporting mitochondrial health and the glutathione system. The multifaceted approach includes treatments, nutrition, sleep, energy management, stress management, and quality of life factors. Treatments may include exogenous glutathione, cysteine containing supplements, micronutrient supplementation, hormone balancing (e.g., thyroid or sex hormones), treatment for specific maladies disrupting homeostasis (e.g., pain, cardiovascular, allergic reaction, infection), heat therapy, bodywork, or acupuncture.

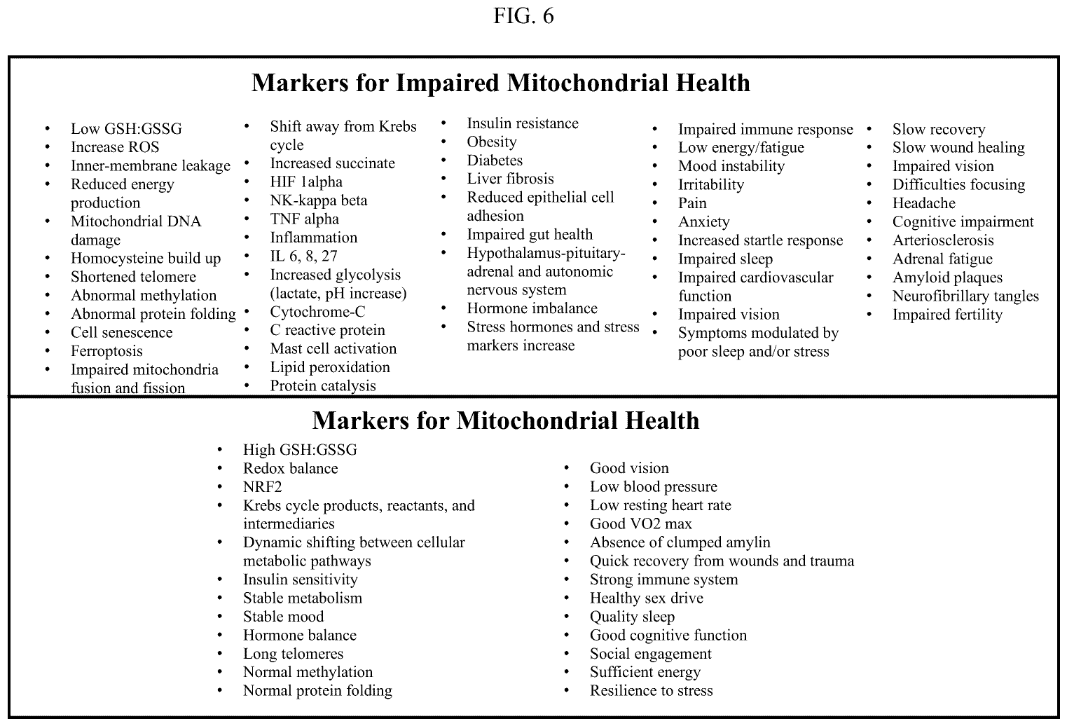

FIG. 6 is a non-exhaustive list of candidate biomarkers to assess mitochondrial function, distinguishing between markers indicative of healthy mitochondrial activity and those suggestive of mitochondrial dysfunction. Healthy markers include normal levels of ATP production and balanced redox status, whereas markers of dysfunction might include elevated ROS levels, mitochondrial DNA damage, indicators of chronic inflammation or altered metabolism, and impaired metabolic enzyme activities.

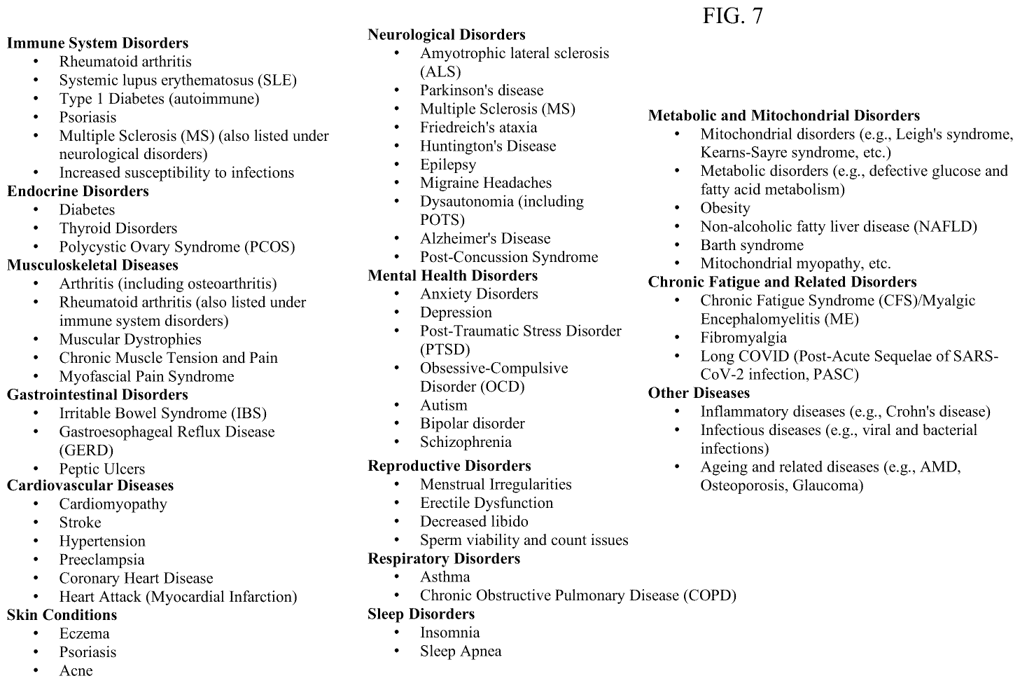

FIG. 7 is a non-exhaustive list of diseases associated with depleted GSH. It covers neurological disorders, metabolic syndromes, cardiovascular diseases, and immune system disorders.

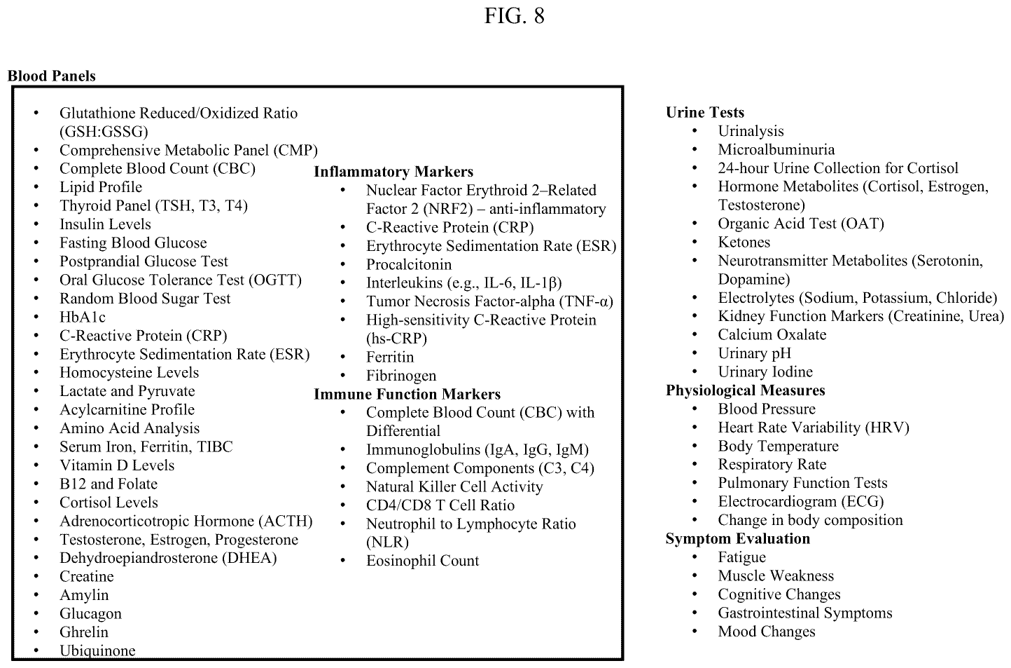

FIG. 8 provides a non-exhaustive list of diagnostic tests to evaluate glutathione and mitochondrial health. It includes specific blood tests (e.g., glutathione levels, complete metabolic panel, oxidative stress markers), urine tests (e.g., organic acid test, hormone metabolites), and other physiological measures (e.g., heart rate variability, respiratory rate) that help assess the functional status of these critical cellular components.

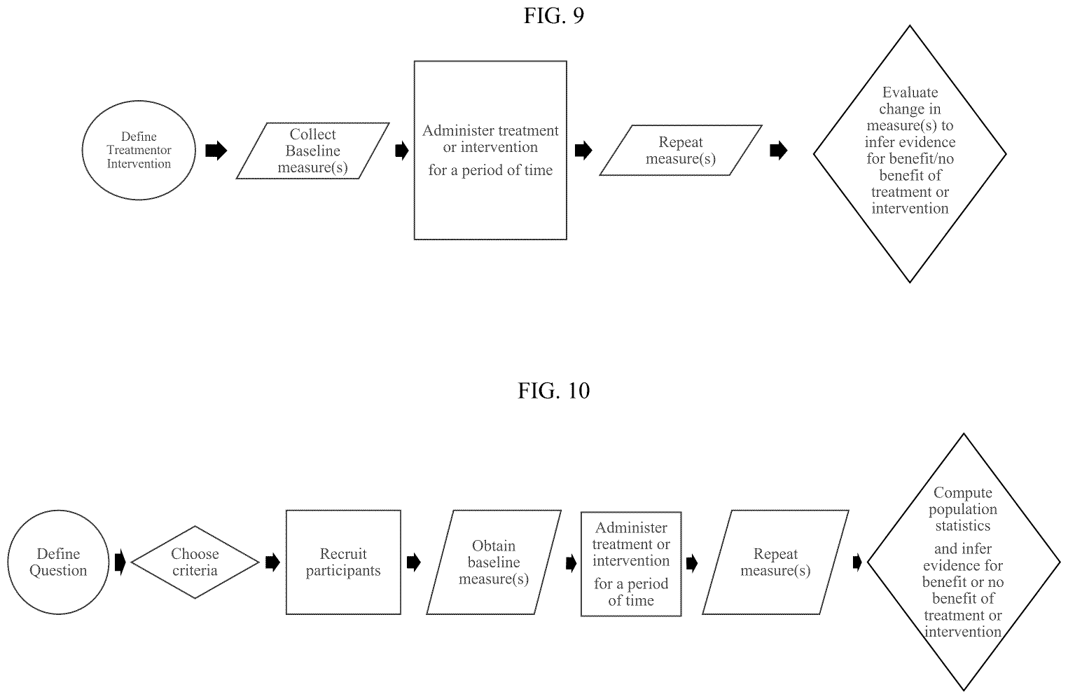

FIG. 9 illustrates a method for assessing the effectiveness of a specific treatment or intervention on an individual level. The depicted process involves: Defining the treatment or intervention, specifying the nature and parameters of the treatment to be tested, collecting baseline measures, gathering initial health metrics or status indicators before the intervention is applied, administering the treatment or intervention, applying the specified treatment over a determined period to allow measurable changes, and repeating measures: collecting the same measures post-treatment to capture the impact of the intervention. The last step involves evaluating changes in measures by analyzing the pre and post-intervention data to determine whether the treatment provided a benefit, indicated by improvement in the measured parameters, or showed no significant change or deterioration, suggesting no benefit.

FIG. 10 expands the individual assessment model (from FIG. 9) to a population level, ideal for broader clinical or scientific validation by defining the research question, setting the scope and objectives of the population study regarding the treatment, choosing criteria for participant recruitment, establishing inclusion and exclusion criteria to select appropriate study participants, obtaining baseline measures: collecting initial data from all participants to establish a control point for future comparison, administering the treatment or intervention: implementing the treatment across the participant group for a consistent duration, repeating measures post-intervention: gathering the same health metrics after the treatment period, and computing population statistics to analyze the collective data, aiming to infer the general effectiveness of the intervention.

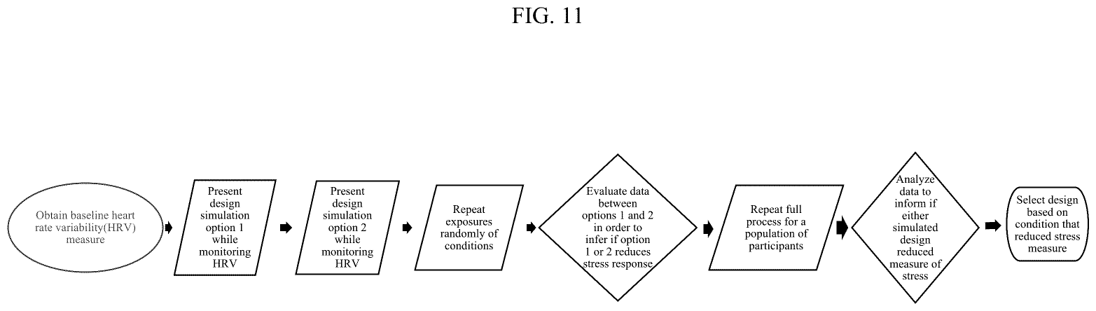

FIG. 11 details a method that focuses on using design elements to influence wellbeing, particularly through stress response management via heart rate variability (HRV) monitoring: measuring initial HRV to establish a reference point for each participant, introducing different environmental or design settings to the participant while continuously monitoring HRV, alternating between design options to avoid bias and gather reliable data, comparing HRV responses to different design simulations to identify which one optimally reduces stress responses, applying the same method across a group to validate which design consistently lowers stress, choosing the design that effectively decreases stress, as evidenced by HRV improvements across the study.

The herein described embodiments and figures are not an exhaustive description, but rather presented to help elucidate the utility of the claims. All presented features can be used in combination or in isolation, and again are examples rather than a complete elaboration of the scope and applications of the set of claims that define the invention.

What is claimed for is:

1) The method of establishing the health status of a tissue, individual, or group comprising of:
a) obtaining a quantitative measure of cellular metabolism using one or more of the following methods i) biological sample analysis, ii) breath analysis, iii) metabolic imaging, iv) cellular metabolic indicators, including but not limited to the ATP cycle, the methylation cycle including but not limited to homocysteine, the Krebs cycle and oxidative phosphorylation, anaerobic glycolysis, the pentose phosphate pathway, lipid peroxidation, protein catabolism, lipolysis and fatty acid oxidation, Cori cycle, ketosis, gluconeogenesis, stress, and v) direct or indirect measures of mitochondrial health including but not limited to any one of the previous categories as well as metabolites linked to hormone production or catabolism, pro- or anti-inflammatory messenger, other modulators of metabolic function, symptoms consistent with mitochondrial dysfunction, energetic level, emotional regulation/dysregulation, stress, or autonomic nervous system state,
b) utilizing the obtained measure to assess health status, including but not limited to: i) biological age, ii) overall risk of disease, iii) selection criteria for one or more treatment, iv) as a method to identify conditions or interventions that impair health or enhance wellbeing, or v) as a metric for evaluating health interventions, including but not limited to determining optimal dose, and/or determining whether a stimuli, real or simulated environment, activity, or design is beneficial for wellbeing.

2) A method as in claim 1, comprising the additional steps:
a) collecting the measure at multiple time points,
b) optionally calculating a measure of change or rate of change normalized by the measure or change at the latest timepoint, and
c) interpreting the change in measures over these time points as an indication of a change in the health status of the tissue, individual, or group.

3) A method as in claim 2, comprising the additional steps:
a. understanding that measures may be affected by fluctuating symptoms, and
b. collecting either qualitative or quantitative measures of pain level or symptoms along with the metabolic measure to control for the variability of pain or symptom severity over the course of an inter-measurement interval.

4) A method as in claim 2, comprising the additional steps:
a) applying one or more health interventions between measures, including but not limited to treatments, stimuli, and/or lifestyle changes, and
b) interpreting the change in measures before and after the health intervention as evidence to support or fail to support the efficacy of the health intervention.

5) A method as in claim 2, with the additional steps comprising:
a) repeating the method for a population of individuals, and
b) pooling the data to inform the efficacy of the design, or health or lifestyle intervention across a population.

6) A method as in claim 1 in which the quantitative measure of cellular metabolism is telomere length, and thus comprising the step in which:
a) realizing that telomere length is a function of the cumulative cellular metabolic status over time,
b) collecting one or more samples, including but not limited to blood and tissue, and
c) measuring the average telomere length in each sample,
d) interpreting the telomere length as a measure of metabolic status over time, with long telomeres corresponding to tissue with a health status suggestive of mitochondrial dysfunction.

7) A method as in claim 1 in which the quantitative measure comprises one or more metabolic measures or imaging methods, including but not limited to:
i) measuring a cellular metabolic metabolite such as succinate, lactate, glucose, homocysteine, ATP, ADP, NADP/H, NAD/H, glutathione, or another redox coupling agent or indicator of redox balance or oxidative or reductive stress; or characterizing the response to the application of one or more doses of an electron donor or precursor,
ii) measuring enzymes including but not limited to: glutathione reductase, glutathione peroxidase, glucose-6-phosphate dehydrogenase, glutathione-s-transferases, and carbohydrate metabolism enzymes,
iii) measuring additional markers of redox imbalance including but not limited to malondialdehyde, superoxide dismutase, 4-hydroxynonenal, oxidative protein products, and toxins including but not limited to Per- and Polyfluoroalkyl Substances,
iv) measuring inflammatory markers, cytokines, and other messenger molecules that are modulated by the levels of reactive oxygen species and/or reduced glutathione either at a single time point or as a dynamic response to a stimuli,
v) evaluating presence of a vitamin, mineral, or amino acid deficiency,
vi) characterizing constituent tissue properties including but not limited to pH, hypoxia, hyperoxia, and mechanical tissue properties, and/or the shape or membrane potential of the mitochondria, and/or
vii) characterizing static or dynamic stress response or immune signaling response, or the systems that modulate the return of that signaling mechanism to pre-stimulus baseline, including but not limited to the pentose phosphate pathway and related metabolic cycles.

8) A method as in claim 1 in which the quantitative measure comprises the additional steps of quantifying the oxygen to carbon dioxide respiratory exchange ratio directly or inferred through measuring respiration or carbon dioxide directly or indirectly.

9) A method as in claim 1 adding the additional steps comprising:
a) recognizing that stress modulates cellular metabolism and when prolonged, stress depletes the reduced glutathione pool which is necessary for maintaining mitochondrial health, and thus
b) measuring one or more stress measurement as an indirect measure to inform whether an applied condition and or intervention is likely to promote or reduce mitochondrial health, with methods including but not limited to: heart rate, heart rate variability, galvanic skin response, blood pressure, respiratory rate, thermal imaging, electroencephalography, electromyography, saliva cortisol, hair cortisol, pupil dilation, changes in voice or facial expression, and changes in behavioral and activity data, including but not limited to changes in sleep, engagement in relaxing activities, reduction in nervous repetitive movements, or self-reported symptoms, mood, dysregulation, or relaxation scales.

10) A method as in claim 1, in which the method of informing health status based on the following steps as used alone or in combination with other metrics:
a) recognizing i) mitochondrial health and dysfunction as a shared bio mechanism across disease states,
ii) glutathione redox status as critical to maintaining mitochondrial health, and
iii) glutathione redox status as an indicator of mitochondrial function/dysfunction,
b) obtaining access to a biological sample, including but not limited to blood or tissue,
c) measuring the reduced glutathione concentration (GSH), or surrogates that incorporate other redox pairs,
d) measuring the oxidized glutathione concentration (GSSG), or surrogates that incorporate other redox pairs,
e) calculating the ratio of reduced to oxidized glutathione (GSH:GSSG) or the inverse (GSSG:GSH), or a surrogate using other redox pairs either with their pairs or across pairs,
f) optionally normalizing the measure by age,
g) interpreting the ratio as a direct indicator of health status.

11) A method as in claim 10 comprising the additional steps of:
a) collecting the data across a population of individuals, and
b) computing the mean of the population,
c) interpreting the health of the population via the mean of the population
e) using the data to guide decisions that can improve the health of a population, and
f) repeating the measures to provide evidence to support or fail to support the hypothesis that a given decision or intervention improves the health status of the population.

12) A method as in claim 11 comprising the additional steps of:
a) computing the standard deviation of the population,
b) interpreting the health disparity across the population via the standard deviation, and
c) using the above metric as a target for guiding decisions and/or policies that improve the health of a population and/or reduce health disparity.

13) A method as in claim 10 comprising the additional steps:
using the metric to estimate an individual's risk for adverse health outcomes, including but not limited to:
cardiovascular event, maternal mortality, post-traumatic stress disorder, mental health crisis, organ failure, infection, cancer initiation, injury, progression, or spreading of cancer, prolonged recovery from a physical trauma, or initiation of a metabolic disorder.

14) A method as in claim 10 for guiding treatment by augmenting the method with the additional steps comprising:
a) measuring the ratio before and after initiation of a treatment for a given individual,
b) optionally calculating a measure of change or rate of change normalized by the measure or change at the latest timepoint,
c) evaluating whether the metric indicates a benefit (either by improving or demonstrating a stabilization of a deteriorating condition), or a failure to provide benefit, and
d) continuing or changing treatment based on the above measurement.

15) A method as in claim 10, wherein the process is used to evaluate the efficacy of a given treatment or lifestyle intervention comprising the additional steps:
a) choosing criteria by which to select individuals for the study, including but not limited to, length of illness, toxin exposure including but not limited to heavy metals and Per- and Polyfluoroalkyl Substances, individuals with one or more diagnosis, self-reported symptoms, genetic mutations including but not limited to GP-6D deficiency, methylation errors, MTHFR and APOE, GFPT2, ERICH1, low GSH:GSSG, one-time administering liposomal glutathione to an individual for screening and including those that have a self-reported or measured benefit, impaired health status as indicated by a blood measure, urine measure, metabolic metric, imaging study, or stress metric for example high saliva cortisol,
b) recruiting participants according to the selection criteria,
c) collecting a baseline GSH:GSSG or surrogate measure for each participant, and optionally additional quantitative and qualitative health metrics,
d) applying one or more treatments or life-style interventions,
e) collecting one or more additional time points of GSH:GSSG or surrogate measure for each participant, and optionally additional quantitative and qualitative health metrics,
f) computing population statistics on the collected dataset, in order to inform the efficacy of the health intervention for the population under study, and optionally infer whether the treatment(s) and/or intervention(s) applied merit further study or use in larger populations.

16) A method as in claim 10 wherein the measure is referred to as the biological age of a tissue or an individual by comprising the additional steps of:
a) understanding that the aging process relates to mitochondrial dysfunction which occurs when stressors fall on tissue which is oxidized (e.g., accumulated reactive oxygen or nitrogen species, under hypoxic conditions, and/or under conditions of inflammation),
b) collecting the data across a large population of individuals of all ages,
c) identifying which chronological age has a mean GSH:GSSG ratio or surrogate which matches that of the individual under question, and
d) inferring the biological age of the individual as the chronological age of the group to which the individual's ratio most closely approximates.

17) A method as in claim 10 in which the method is used to ensure that the side effects of a treatment do not degrade the overall health of an individual, comprising the additional steps:
a) obtain a baseline GSH:GSSG ratio or surrogate prior to beginning a treatment,
b) obtaining GSH:GSSG ratio or surrogate during the treatment regimen to monitor GSH:GSSG and optionally collecting patient symptoms and reports of medically significant events in order to help differentiate life events from medication side effects that may affect the measure,
c) interpreting changes in GSH:GSSG ratio or surrogate, optionally aided by patient reports, to guide continuation of treatment, altering of does, or cessation, and optionally
d) obtaining GSH:GSSG ratio or surrogate measure to inform whether dose changing or cessation increases GSH:GSSG ratio.

18) A method as in claim 1 comprising additional steps:
a) collecting one or more measure of cellular metabolism, including but not limited to imaging or sample characterization, and
b) measuring one or more measures of cellular metabolism or stress, including but not limited to respiratory exchange rate, heart rate and/or heart rate variability, respiration variability, and
c) establishing the use of the latter measure as a surrogate for the former measure as a means of directly or indirectly informing health status or change of health status due to an intervention or presented stimuli.

19) A method as in claim one, where metabolic measures are used to tailor individual dose, comprising
a) first taking one or more direct or indirect measure of health status, including but not limited to patient reported energy level, or measurements of glutathione system, hormone levels, and cellular metabolism, which can be done in person, remotely, or via a combination,
b) second, applying a treatment protocol appropriate to the individual, c) third, repeating the measurement(s), and
d) finally, adjusting the dosing of one or more component of the treatment, and repeating measurements until desired endpoint is achieved, including but not limited to energetic homeostasis, or well-being of the individual.

20) A method as in claim one, with the aim of supporting mitochondrial health and/or wellbeing, comprising the additional step of providing a treatment, condition, or life-style intervention aimed at improving health status by supporting key metabolic processes, including but not limited to the glutathione system, hormonal balance, redox balance, and the Krebs cycle.
